(19) United States
(12) Patent Application Publication
(10) Pub. No.: US 2016/0128628 A1
(43) Pub. Date: May 12, 2016

(54) PROJECTED CAPACITIVE DETECTING SYSTEM FOR DETECTING HUMAN ACTIVITIES

(71) Applicant: Yu-Han CHEN, Tainan City (TW)

(72) Inventor: Yu-Han CHEN, Tainan City (TW)

(21) Appl. No.: 14/931,526

(22) Filed: Nov. 3, 2015

(30) Foreign Application Priority Data
Nov. 7, 2014 (TW) 103138660

(51) Int. Cl. A61B 5/00; A61B 5/113; A61B 5/08

(52) U.S. Cl. CPC A61B 5/4815; A61B 5/0816; A61B 5/113; A61B 5/4812; A61B 5/742; A61B 5/6892

(57) ABSTRACT

A projected capacitive detecting system comprises a sensing pad device and a monitoring module. The sensing pad device comprises at least one sensing array unit and at least one control unit. The at least one sensing array unit comprises multiple sensing electrodes arranged in an array. The at least one control unit is electrically connected to the multiple sensing electrodes to receive capacitances of the multiple sensing electrodes. The at least one control unit performs a capacitance conversion to convert the capacitances of the multiple sensing electrodes to obtain multiple capacitive sensing values and digitize the multiple capacitive sensing values to obtain Boolean true/false results. The monitoring module is electrically connected to the at least one control unit to receive and display the multiple capacitive sensing values and the Boolean true/false results as references for determining human activities.

### CROSS-REFERENCE TO RELATED APPLICATIONS

This application claims the benefit of Taiwan patent application No. 103138660, filed on Nov. 7, 2014, the disclosure of which is incorporated herein in its entirety by reference.

### BACKGROUND OF THE INVENTION

1. Field of the Invention

The present invention relates to a detecting system, and more particularly to a projected capacitive detecting system for detecting human activities.

2. Description of Related Art

Polysomnography (hereinafter referred to as PSG) instrument is a medical instrument for recording a user's physiological activities while the user is sleeping, wherein the physiological activities may include brain activity, eyeball activity, muscle activity, breathing status, heart rate, and so on. Hence, the physiological activities recorded by the PSG instrument are used for sleep quality assessment by medical staff. Breathing status is the most significant index for sleep quality evaluation.

When measuring the user's breathing status by PSG, the user has to lie on a bed and an elastic strap equipped with a detector is tightly fastened on the user's chest. User's activity is restricted by the elastic strap, such that the user may not move or turn over casually. When the user inhales, the user's thoracic volume expands to extend the elastic strap. When the user exhales, the user's thoracic volume contracts to shorten the elastic strap. Hence, a detecting result of the detector represents an expansion or a contraction of the user's thoracic volume. Besides, a nasal cannual is mounted on the user's face and nose. The nasal cannual may detect the air pressure when the user is inhaling or exhaling.

As mentioned above, detecting results of the detector mounted on the elastic strap and the nasal cannual are provided to medical staff for sleep quality assessment. However, user's activities is restricted by the attached elastic strap and nasal cannual. That may interrupt the user's sleep and affect the detecting results. In addition, due to personal hygiene concerns, the nasal cannual is disposable, which increases system cost.

### SUMMARY OF THE INVENTION

An objective of the present invention is to provide a projected capacitive detecting system for detecting human activities according to capacitance interaction without wearing a detector.

The projected capacitive detecting system of the present invention comprises a sensing pad device and a monitoring module.

The sensing pad device comprises at least one sensing array unit and at least one control unit. The at least one sensing array unit comprises multiple sensing electrodes arranged in an array. The at least one control unit is electrically connected to the multiple sensing electrodes to receive capacitances of the multiple sensing electrodes. The at least one control unit performs a capacitance conversion to convert the capacitances of the multiple sensing electrodes to obtain multiple capacitive sensing values and digitize the multiple capacitive sensing values to obtain Boolean true/false results.

The monitoring module is electrically connected to the at least one control unit to receive and display the multiple capacitive sensing values and the Boolean true/false results as references for determining human activities.

The user may spread a bed sheet or a mattress protector over the sensing pad device due to comfort and visual acceptability, such that the user can lie on the bed sheet or the mattress protector over the sensing pad device. The present invention detects the user's movement according to capacitance interaction between the sensing pad device and the user's body. The capacitive sensing values and the Boolean true/false results may be references for determining the user's activities.

Compared with the related art as mentioned above, the user would not wear any detector on the user's body when the user is examined by the detecting system of the present invention, such that the present invention would not limit the user's behavior. Therefore, the user may feel more comfortable when the user is examined by the detecting system of the present invention. The interference with and inconvenience of the user in the examination is reduced. The detecting results of the present invention would be highly reliable. Moreover, there is no direct contact between sensing electrodes and user skin which satisfying personal hygiene requirements.

### BRIEF DESCRIPTION OF THE DRAWINGS

FIG. 1 is a circuit block diagram of an embodiment of a projected capacitive detecting system of the present invention;

FIG. 2 is an operational view of a sensing pad device of the projected capacitive detecting system of the present invention;

FIG. 3A is a schematic view of an embodiment of a sensing electrode of the sensing pad device;

FIG. 3B is a schematic view of another embodiment of sensing electrodes of the sensing pad device;

FIG. 4 is a schematically cross-sectional view of FIG. 2;

FIG. 5 is a waveform diagram of capacitances of the sensing electrodes located on a fourth column of the sensing array unit corresponding to the embodiment of FIG. 1 and FIG. 2;

FIG. 6 is a waveform diagram including a first waveform U of the relative capacitive values of the present invention, a second waveform V produced by a conventional polysomnogram (PSG), and an air pressure waveform W produced by a conventional nasal cannual;

FIG. 7 is a waveform diagram of the relative capacitive value of the sensing electrode of the present invention;

FIG. 8 is a waveform diagram of noise; and

FIG. 9 is a waveform diagram of combined waveforms, wherein the waveforms of the relative capacitive values of FIG. 7 are respectively combined with the noise of FIG. 8.

### DETAILED DESCRIPTION OF THE PREFERRED EMBODIMENT

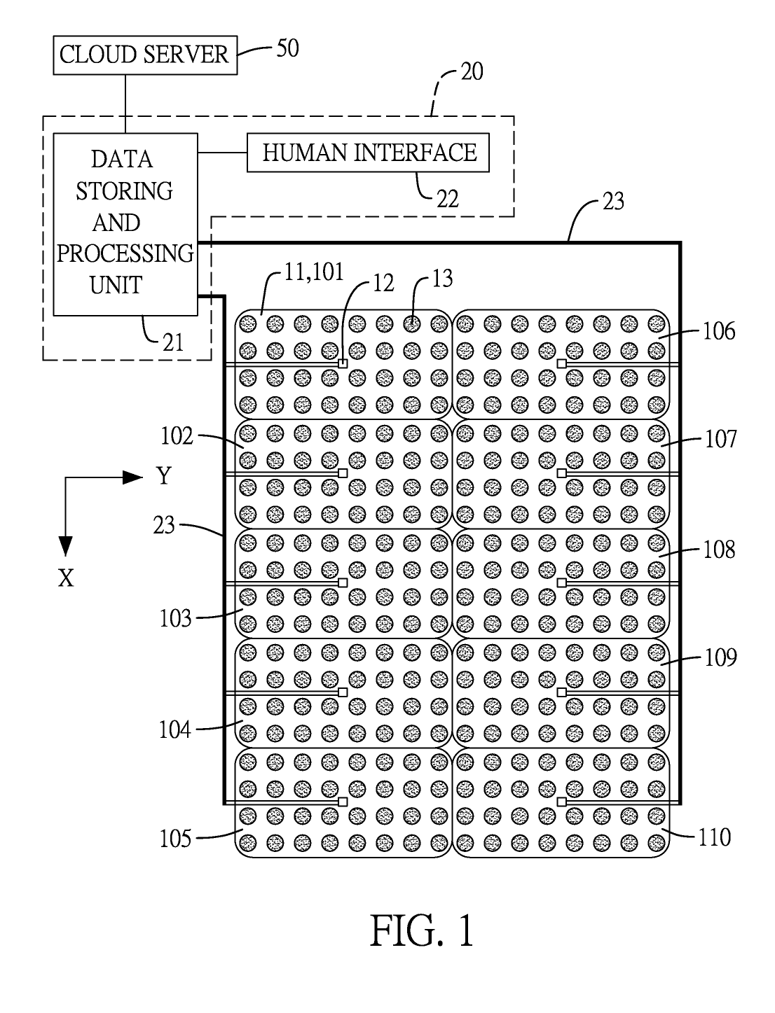

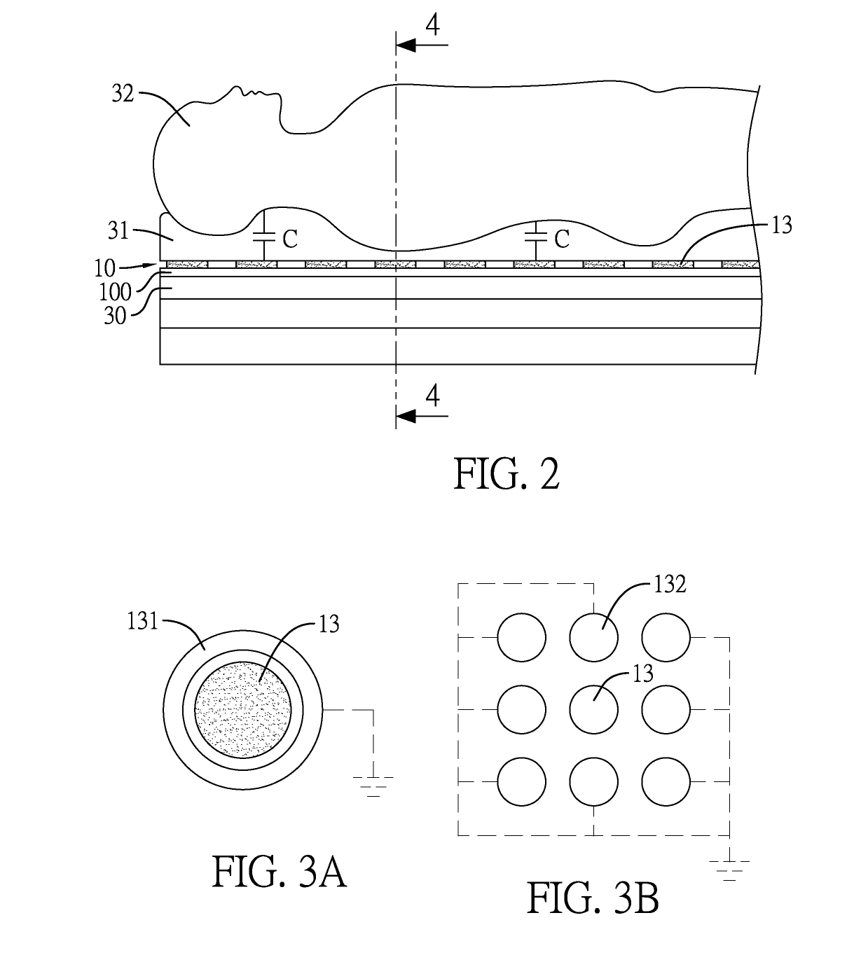

With reference to FIG. 1 and FIG. 2, a detecting system of the present invention comprises a sensing pad device 10 and a monitoring module 20. The sensing pad device 10 can be spread over a mattress 30. Bedding 31, such as a bed sheet or a mattress protector, can be put on the sensing pad device 10, such that a user 32 can lie on the bedding 31 over the sensing pad device 10.

The sensing pad device 10 comprises at least one sensing array unit 11, at least one control unit 12, and a flexible pad 100. The control unit 12 may be a single chip. The sensing array unit 11 and the control unit 12 may be disposed on a surface of the flexible pad 100. The sensing array unit 11 comprises multiple sensing electrodes 13 arranged in an array. The sensing electrodes 13 may be electrical conductive sheets. The sensing electrodes 13 are disposed between the bedding 31 and the flexible pad 100. When the user 32 lies on the bedding 31, the user's body and the sensing electrodes 13 are separated by the bedding 31, such that the user's body would not directly contact the sensing electrodes 13.

The control unit 12 is electrically connected to the sensing electrodes 13 and outputs a physical quantity, such as a current, to the sensing electrodes 13. Meanwhile, the control unit 12 samples capacitance of each one of the sensing electrodes 13 at a sampling rate (or sampling cycle) and performs an analog-to-digital (A/D) conversion (capacitance conversion) to convert the capacitances of the sensing electrodes 13 to physical features, such that an A/D conversion result of the capacitance of each one of the sensing electrodes 13 is a capacitive sensing value C. When there is no person lying on the bedding 31, the capacitive sensing values C produced by the control unit 12 are constant.

Since the human body is electrically conductive, a capacitance approximating 200 pF exists between the human body and earth. When the user 32 lies on the bedding 31, charge distribution of the sensing electrodes 13 not below the user's body may not have an obvious change, and charge distribution of the sensing electrodes 13 below the user's body may have obvious changes, such that the capacitive sensing values C of the sensing electrodes 13 below the user's body accordingly change obviously. Based on a capacitance expression C=ε×A/d, the capacitance variation varies with a dielectric variation or a distance variation. Because a pressure variation occurs on the sensing pad device 10 pressed by the user's body, the dielectric and the distance between the user's body and the sensing electrodes 13 change accordingly to induce the dielectric variation and the distance variation. Hence, the position of the human body over the sensing pad device 10 may be derived by referring to the sensing electrode 13 that has the capacitive sensing value C being varied.

The control unit 12 stores the capacitive sensing values C. Resolution of the capacitive sensing value C of each one of the sensing electrodes 13 may be 16 bits. In order to simplify calculation, the control unit 12 subtracts a base value from the capacitive sensing value C of each one of the sensing electrodes 13 to obtain a relative capacitive value ΔC. For example, the base value may be 15020 and the capacitive sensing values C may include 15023, 15025, 15028, 15026, 15022, and 15023, such that the corresponding relative capacitive values ΔC are 3, 5, 8, 6, 2, and 3. Hence, when the control unit 12 processes the relative capacitive values ΔC instead of the capacitive sensing values C, the calculation for the relative capacitive values ΔC is simplified and may be speeded up. The sampling rate may be higher than a normal breathing frequency (0.05˜0.5 Hz) of a human. For example, the sampling rate may be 3 Hz (or sampling cycle may be 0.33 seconds).

The control unit 12 further digitizes the capacitive sensing values C after receiving the capacitive sensing values C. In other words, each one of the capacitive sensing values C may correspond to a Boolean true/false result. The Boolean true/false result may represent a first logic level or a second logic level complementary to the first logic level. For example, when the first logic level is 1, the second logic level is 0. Hence, data quantity of each one of the Boolean true/false results of the capacitive sensing values C is just one bit.

When the user lies over the sensing pad device 10, some of the sensing electrodes 13 below the user are pressed, and the remaining sensing electrodes 13 are unpressed. The capacitive sensing values C of the sensing electrodes 13 that have been pressed are higher than the capacitive sensing values C of the unpressed sensing electrodes 13. The control unit 12 determines the capacitive sensing value C higher than a reference value as logic 1 and determines the capacitive sensing value C lower than the reference value as logic 0. Therefore, the sensing electrode 13 whose capacitive sensing value C is determined to be logic 1 may be pressed by the user.

The monitoring module 20 performs data input and data output via a human interface, and communicates with external systems. With reference to FIG. 1, the monitoring module 20 comprises a data storing and processing unit 21 and a human interface 22. The data storing and processing unit 21 is electrically connected to the at least one control unit 12 via at least one bus 23 to receive and store the capacitive sensing value C, the relative capacitive value ΔC, and the Boolean true/false result of each one of the sensing electrodes 13 calculated by the at least one control unit 12 at each one of the sampling cycles. The human interface 22 may be a display. The human interface 22 is electrically connected to the data storing and processing unit 21 to display the sensing results and calculating results, such as the capacitive sensing value C, the relative capacitive value ΔC, and the Boolean true/false result of each one of the sensing electrodes 13. The capacitive sensing values C or the relative capacitive values ΔC of the sensing electrodes 13 are adapted to examine micro activities, such as breathing, of the user lying over the sensing pad device 10. The Boolean true/false results are adapted to examine a body motion and posture of the user lying over the sensing pad device 10. With the increased number of the buses 23, the data transmitting quantity, sampling speed, and communication efficiency may be increased.

In order to improve the capacitance sensing accuracy and exclude external noise interference, with reference to FIG. 3A, a ring electrode 131 may be disposed around each one of the sensing electrodes 13. The ring electrode 131 is electrically connected to a discharging path, such as a ground as shown in FIG. 3A. Hence, noises would be grounded via the discharging path. The noise interference would be decreased. In another embodiment, with reference to FIG. 3B, nine sensing electrodes are defined as a set. When the control unit 12 is going to perform the A/D conversion for the capacitive sensing value C of the sensing electrode 13, the data storing and processing unit 21 may connect the eight sensing electrodes 132 surrounding the sensing electrode 13 to the discharging path, such that the sensing electrode 13 at the center is shielded from noise.

A first method to determine the user's breathing activity is disclosed as follows. The Boolean true/false results of the sensing electrodes 13 below the user are determined to be the first logic level, and the Boolean true/false results of the remaining sensing electrodes 13 not below the user are determined to be the second logic level. The data storing and processing unit 21 filters the capacitive sensing values C of the sensing electrodes 13 which are below the user's chest and belly and are pressed or completely covered by the user, and are determined to be the first logic level by a band-pass filtering process to obtain multiple filtered results. A specific band-pass frequency band corresponds to human breathing frequency, such as between 0.05 and 0.5 Hz. The filtered results of the capacitive sensing values C may be displayed on the human interface 22 for medical staff to review.

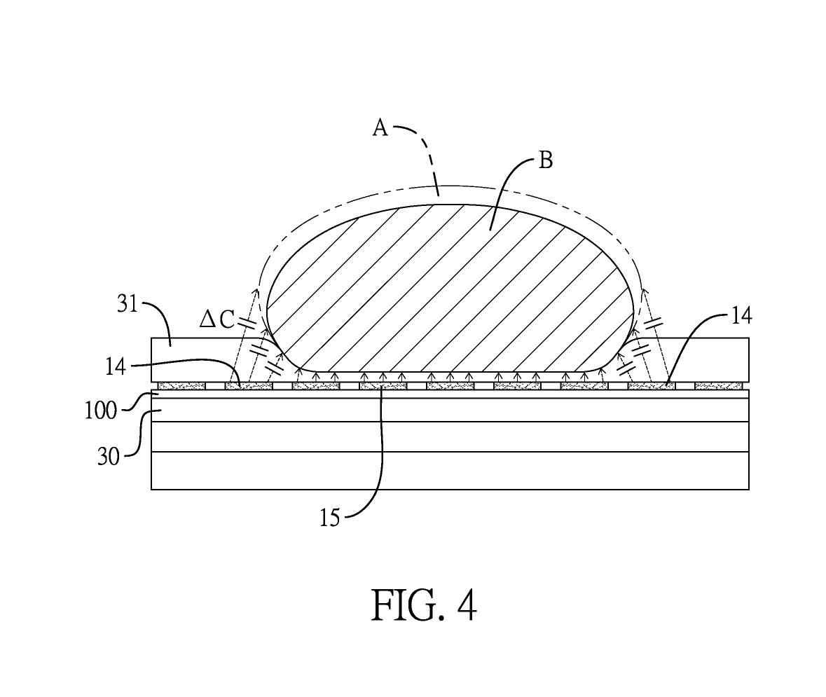

A second method to determine the user's breathing activity is disclosed as follows. With reference to FIG. 4, the Boolean true/false results of the sensing electrodes 13 unpressed by the user are determined to be the second logic level, and the Boolean true/false results of the remaining sensing electrodes 13 pressed by the user are determined to be the first logic level. The data storing and processing unit 21 will not process the relative capacitive values ΔC of the sensing electrodes 15 determined to be the first logic level. The data storing and processing unit 21 performs signal quality estimation to the relative capacitive values ΔC of the sensing electrodes 15 determined to be the second logic level to determine the sensing quality of such relative capacitive values ΔC. For example, signal-to-noise ratio (SNR) is an index for the data storing and processing unit 21 to determine the sensing quality. Further, the data storing and processing unit 21 takes at least one relative capacitive value ΔC that has better sensing quality as reference to determine the user's breathing activity. When the data storing and processing unit 21 performs the signal quality estimation, the data storing and processing unit 21 calculates a positive slope or a negative slope of the at least one relative capacitive value ΔC and determines times of slope variations (the positive slope changes to the negative or the negative slope changes to the positive slope) of the at least one relative capacitive value ΔC. Hence, the data storing and processing unit 21 selects the relative capacitive value ΔC that has the fewest times of slope variations or selects multiple relative capacitive values ΔC that have fewer times of slope variations than a threshold number of times of slope variations, such that the selected relative capacitive values ΔC are displayed on the human interface 22.

The mechanism of the variations between the positive slope and the negative slope of the relative capacitive values ΔC is disclosed as follows. With reference to FIG. 4, a sectional schematic view of a user's thoracic cavity corresponding to FIG. 2 is disclosed. Regarding the sensing electrodes 14 that are not below the user's body and are unpressed by the user (such as the sensing electrodes below the user's chest and belly and determined to be the second logic level adjacent to the sensing electrodes determined to be the first logic level), when the user inhales, a volume of the inhaling thoracic cavity A is expanded, such that distances between surfaces of two opposite sides of the user's thoracic cavity and the sensing electrodes 14 are gradually decreased, and the relative capacitive values ΔC of such sensing electrodes 14 are correspondingly and gradually increased. Therefore, the relative capacitive values ΔC of such sensing electrodes 14 tends to be increased. Inversely, when the user exhales, the volume of the exhaling thoracic cavity B contracts, such that distances between surfaces of two opposite sides of the user's thoracic cavity and the sensing electrodes 14 are gradually increased, and the relative capacitive values ΔC of such sensing electrodes 14 are correspondingly and gradually decreased. Therefore, the relative capacitive values ΔC of such sensing electrodes 14 tends to be decreased. In brief, the relative capacitive value ΔC corresponding to inhaling thoracic cavity tends to be increased and the relative capacitive value ΔC corresponding to exhaling thoracic cavity tends to be decreased. As a result, regarding the sensing electrodes 14 not below the user's body and are unpressed by the user, their relative capacitive values ΔC varies with the inhaling or exhaling thoracic cavity.

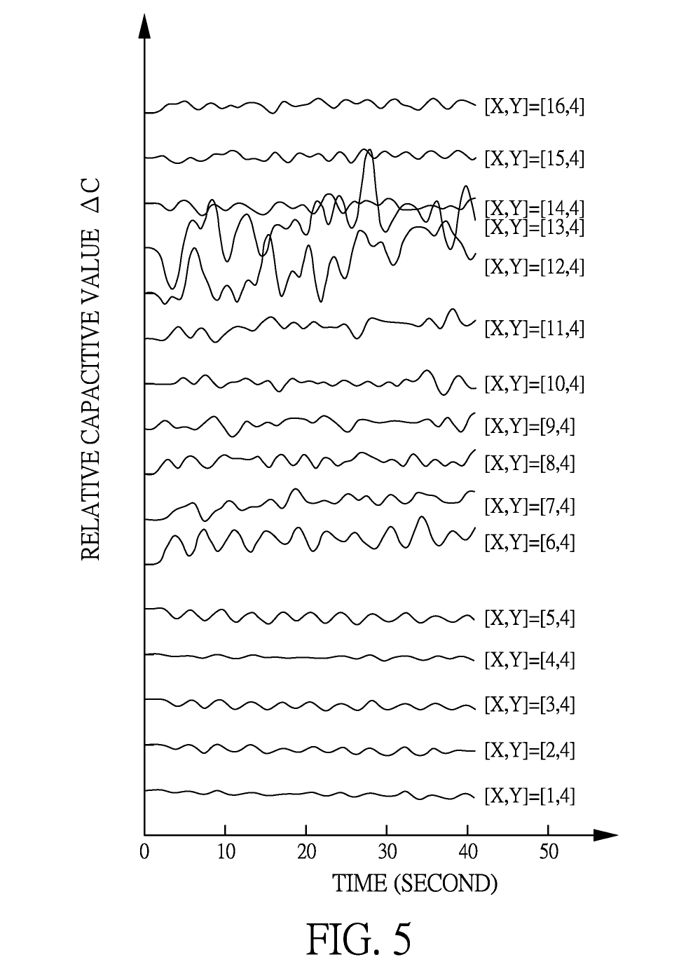

The data storing and processing unit 21 produces a waveform diagram of the relative capacitive values ΔC verse time and transmits the waveform diagram to the human interface 22, such that the human interface 22 displays the waveform diagram. With reference to FIG. 5, the waveform diagram shows waveforms of the relative capacitive values ΔC of the sensing electrodes 13 that are disposed in the fourth column of the sensing array unit 11 as shown in FIG. 1. The waveform diagram is for the data storing and processing unit 21 to determine the user's breathing status.

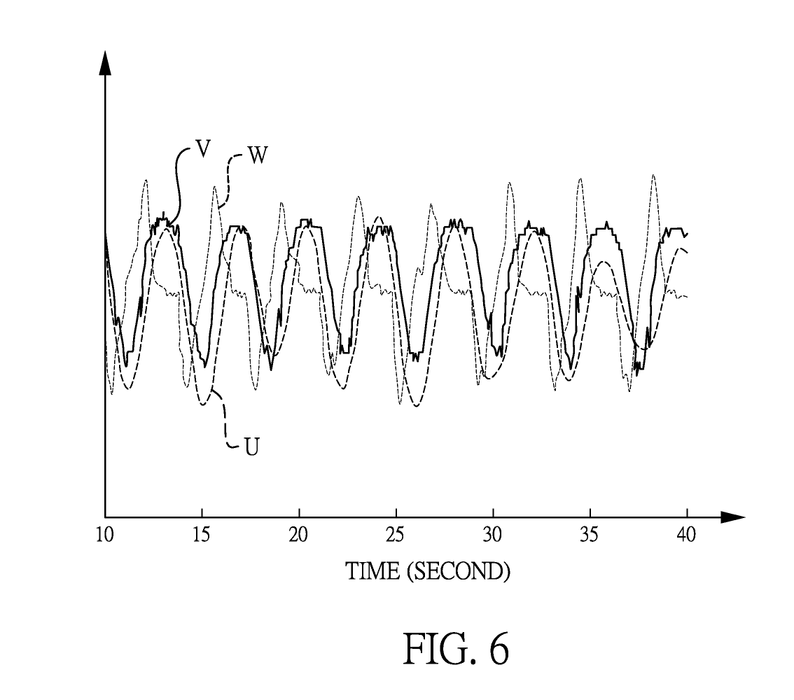

With reference to FIG. 5, the waveform of the relative capacitive values ΔC of the sensing electrodes 13 located in coordinates [5,4] is a regular waveform. With further reference to FIG. 6, FIG. 6 shows a first waveform U of the relative capacitive values ΔC of the present invention, a second waveform V produced by a conventional polysomnogram (PSG), and an air pressure waveform W produced by a conventional nasal cannual. The first waveform U, the second waveform V, and the air pressure waveform W have high similarity (higher than 88%). Hence, the first waveform U of the present invention is sufficient for the data storing and processing unit 21 to determine the user's breathing status.

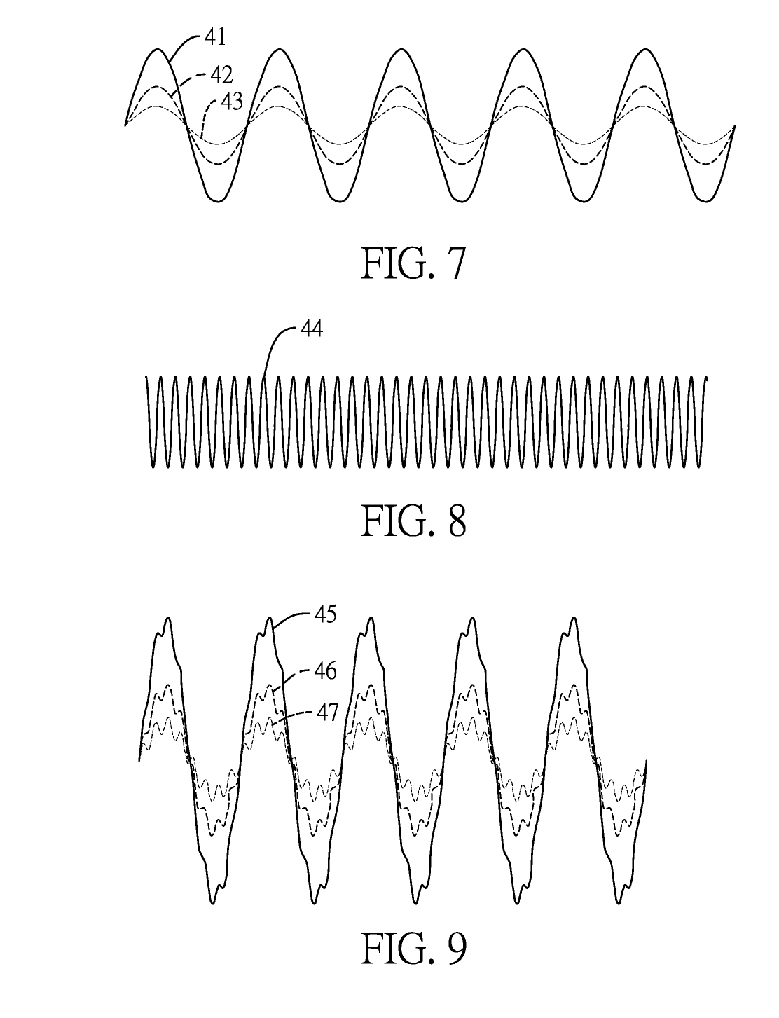

With reference to FIG. 5, the relative capacitive values ΔC of the sensing electrodes 13 located in coordinates [5,4] has fewer or the fewest times of slope variations. In other words, among the sensing electrodes 14 whose Boolean true/false results are determined to be the second logic level, the sensing electrodes 14 therein most approximating the user's body has the highest relative capacitive value ΔC, such that the sensing electrodes 14 have fewer times of slope variations to form the regular waveform. With reference to FIG. 7, a waveform 41 represents the relative capacitive value ΔC of the sensing electrode 14 most approximate to the user, and remaining waveforms 42, 43 respectively represent two relative capacitive values ΔC of any two of the sensing electrodes 14 distal to the user. The amplitude of the waveform 41 is higher than the amplitudes of the remaining waveforms 42, 43. When the waveforms 41, 42, 43 are affected by a same noise 44 as shown in FIG. 8, the waveforms 41,42,43 would be combined with the noise 44 to form a first combined waveform 45, a second combined waveform 46, and a third combined waveform 47 as shown in FIG. 9. With reference to FIG. 9, during a unit of time, the first combined waveform 45 has fewer times of slope variations than the second combined waveform 46 and the third combined waveform 47 respectively. As a result, after the data storing and processing unit 21 calculates the times of slope variations of each one of the relative capacitive values ΔC, the sensing electrodes 14 whose relative capacitive values ΔC are determined to have fewer times of slope variations are most approximate to the user. The estimation method as mentioned above may determine the sensing electrodes 14 that are most approximate to the user, and the waveforms of the capacitive sensing values C of such sensing electrodes 14 are formed in regularity and may be reference for determining the user's breathing activity.

Besides the determination of the user's breathing activity, the present invention may determine the user's body movement and simple posture. The data storing and processing unit 21 may calculate a total number of the sensing electrodes 13 determined to be the first logic level according to the Boolean true/false results and determine whether the total number is higher than a threshold value. When the user lies supine over the sensing pad device 10, the user has wider area over the sensing pad device 10, such that more Boolean true/false results of the capacitive sensing values C of the sensing electrodes 13 are determined to be the first logic level, and the total number of the sensing electrodes determined to be the first logic level would be higher than the threshold value. Accordingly, the data storing and processing unit 21 determines that the user lies supine over the sensing pad device 10. Inversely, when the user lies on his or her side over the sensing pad device 10, the user has smaller area over the sensing pad device 10, such that fewer Boolean true/false results of the capacitive sensing values C of the sensing electrodes 13 are determined to be the first logic level, and the total number of the sensing electrodes determined to be the first logic level would be lower than the threshold value. The data storing and processing unit 21 then determines that the user lies on his/her side over the sensing pad device 10.

In addition, the data storing and processing 21 may determine a number of times, sequence, and difference of the user's turning over and movement as reference to determine the user's activity, sleep quality, sleep stage, and so on.

With reference to FIG. 1, ten sensing array units are disclosed as an example. The ten sensing array units 101-110 include a first sensing array unit 101, a second sensing array unit 102, a third sensing array unit 103, a fourth sensing array unit 104, a fifth sensing array unit 105, a sixth sensing array unit 106, a seventh sensing array unit 107, an eighth sensing array unit 108, a ninth sensing array unit 109, and a tenth sensing array unit 110 that are at positions corresponding to the user's different body portions respectively. When the user lies over the sensing pad device 10, the first sensing array unit 101, the second sensing array unit 102, the third sensing array unit 103, the sixth sensing array unit 106, the seventh sensing array unit 107, and the eighth sensing array unit 108 are at positions corresponding to the user's upper body (such as the chest). The data storing and processing unit 21 may perform the breathing estimation as mentioned above based on the first sensing array unit 101, the second sensing array unit 102, the third sensing array unit 103, the sixth sensing array unit 106, the seventh sensing array unit 107, and the eighth sensing array unit 108. The remaining sensing array units including the fourth sensing array unit 104, the fifth sensing array unit 105, the ninth sensing array unit 109, and the tenth sensing array unit 110 are at positions corresponding to the user's lower limbs (such as feet) that is irrelative to breathing activity, such that the data storing and processing unit 21 neglects the rest sensing array units as listed above to reduce data processing quantity. Moreover, the sensing array units 101-110 are at positions respectively corresponding to different portions of the user's body. For example, the third sensing array unit 103 and the eight sensing array unit 108 may be at positions corresponding to the user's left hand and right hand respectively. The fifth sensing array unit 105 and the tenth sensing array unit 110 may be at positions corresponding to the user's left foot and right foot respectively. The data storing and processing unit 21 may determine whether the user's body portions move according to the total number of the sensing electrodes 13 determined as the first logic level or the second logic level in the sensing array units 101-110.

The second method to determine the user's breathing activity is described as follows. When the user lying on the bedding 31 moves or turns the body over, the relative capacitive values ΔC of some of the sensing electrodes 13 may change, such that the sensing electrodes 13 for determining the user's breathing activity change. In this situation, the data storing and processing unit 21 further calculates a difference between a total number of the sensing electrodes 13 determined to be the first logic level in a present sampling cycle and a total number of the sensing electrodes 13 determined to be the first logic level in a previous sampling cycle. When the user moves or turns the body over, the data storing and processing unit 21 may determine the difference is higher than a predetermined threshold, such that the data storing and processing unit 21 stops performing a present timekeeping and starts to perform a new timekeeping. When the data storing and processing unit 21 determines that a time of static condition is longer than a threshold time while performing the timekeeping, the data storing and processing unit 21 determines the times of slope variations and produces the waveform diagram of the relative capacitive values ΔC as mentioned above. In general, the threshold time is longer than two cycles of human breathing, such as twenty seconds. The static condition as mentioned above represents that the user motionlessly lies on the bedding 31. Hence, the time of static condition represents a time of the user motionlessly lying on the bedding 31.

With reference to the embodiment shown in FIG. 1, the number of the sensing electrodes 13 is three hundred and twenty. The data quantity of the capacitive sensing value C of each one of the sensing electrodes 13 may be 16 bits. Hence, the total data quantity of the capacitive sensing values C received by the data storing and processing unit 21 from all of the sensing electrodes 13 in every sampling cycle is just 5120 bits (or 640 Bytes). The total data quantity of the Boolean true/false results received by the data storing and processing unit 21 from all of the sensing electrodes 13 in every sampling cycle is just 320 bits (or 40 Bytes). Further, the user lying over the sensing pad device 10 of the present invention can move occasionally, not like that the conventional PSG using straps and a nasal cannual that may limit the user's movement. Therefore, the user may feel more comfortable when the user is examined by the detecting system of the present invention. As shown in FIG. 1, the monitoring module 20 may further communicate with a cloud server 50 to upload and backup the sensing data and calculating results to the cloud server 50, such that the cloud server 50 may make use of the sensing data and calculating results to perform remote monitoring and health management.

What is claimed is:

1. A projected capacitive detecting system for detecting human activities, the projected capacitive detecting system comprising:
a sensing pad device comprising:
at least one sensing array unit comprising multiple sensing electrodes arranged in an array;
at least one control unit electrically connected to the multiple sensing electrodes to receive capacitances of the multiple sensing electrodes; and
the at least one control unit performing a capacitance conversion to convert the capacitances of the multiple sensing electrodes to obtain multiple capacitive sensing values and digitize the multiple capacitive sensing values to obtain Boolean true/false results; and
a monitoring module electrically connected to the at least one control unit to receive and display the multiple capacitive sensing values and the Boolean true/false results as references for determining human activities.

2. The projected capacitive detecting system as claimed in claim 1, wherein when the at least one control unit digitizes each one of the multiple capacitive sensing values, the at least one control unit determines each one of the capacitive sensing values higher than a reference value as a first logic level and determines each one of the capacitive sensing values lower than the reference value as a second logic level complementary to the first logic level.

3. The projected capacitive detecting system as claimed in claim 2, wherein the monitoring module calculates a total number of the sensing electrodes determined to be the first logic level and determines whether the total number is higher than a threshold value;
when the total number is higher than the threshold value, the monitoring module determines that a human lies supine; when the total number is lower than the threshold value, the monitoring module determines that the human is in a side-lying position;
the at least one sensing array unit of the sensing pad device includes multiple sensing array units respectively representing different body portions of the human, such that the monitoring module determines movements of the different body portions of the human according to the Boolean true/false results of the multiple sensing array units.

4. The projected capacitive detecting system as claimed in claim 3, wherein the monitoring module calculates a number of times of turning over and movement of the human as references to determine the human's activity, sleep quality, and sleep stage.

5. The projected capacitive detecting system as claimed in claim 3, wherein the monitoring module filters the capacitive sensing values of the sensing electrodes determined to be the first logic level by a band-pass filtering process to obtain multiple filtered results corresponding to a human breathing frequency.

6. The projected capacitive detecting system as claimed in claim 3, wherein the at least one control unit subtracts a base value from the capacitive sensing value of each one of the sensing electrodes to obtain a relative capacitive value;
the monitoring module receives the relative capacitive values, selects the relative capacitive values of the sensing electrodes determined to be the second logic level, calculates times of slope variations of the relative capacitive values, and selects the relative capacitive value that has the fewest times of slope variations or selects multiple capacitive values that have fewer times of slope variations than a threshold number of times of slope variations for determining the human's breathing activity.

7. The projected capacitive detecting system as claimed in claim 1, wherein the monitoring module is electrically connected to the at least one control unit via at least one bus.

8. The projected capacitive detecting system as claimed in claim 2, wherein the monitoring module is electrically connected to the at least one control unit via at least one bus.

9. The projected capacitive detecting system as claimed in claim 3, wherein the monitoring module is electrically connected to the at least one control unit via at least one bus.

10. The projected capacitive detecting system as claimed in claim 6, wherein the monitoring module is electrically connected to the at least one control unit via at least one bus.

11. The projected capacitive detecting system as claimed in claim 7, wherein a plurality of the sensing electrodes surrounding one of the sensing electrodes is electrically connected to a discharging path.

12. The projected capacitive detecting system as claimed in claim 8, wherein a plurality of the sensing electrodes surrounding one of the sensing electrodes is electrically connected to a discharging path.

13. The projected capacitive detecting system as claimed in claim 9, wherein a plurality of the sensing electrodes surrounding one of the sensing electrodes is electrically connected to a discharging path.

14. The projected capacitive detecting system as claimed in claim 10, wherein a plurality of the sensing electrodes surrounding one of the sensing electrodes is electrically connected to a discharging path.

15. The projected capacitive detecting system as claimed in claim 11, wherein the monitoring module communicates with a cloud server to upload and backup the multiple capacitive sensing values and the Boolean true/false results to the cloud server.

16. The projected capacitive detecting system as claimed in claim 12, wherein the monitoring module communicates with a cloud server to upload and backup the multiple capacitive sensing values and the Boolean true/false results to the cloud server.

17. The projected capacitive detecting system as claimed in claim 13, wherein the monitoring module communicates with a cloud server to upload and backup the multiple capacitive sensing values and the Boolean true/false results to the cloud server.

18. The projected capacitive detecting system as claimed in claim 14, wherein the monitoring module communicates with a cloud server to upload and backup the multiple capacitive sensing values and the Boolean true/false results to the cloud server.

19. The projected capacitive detecting system as claimed in claim 17, wherein the monitoring module comprises:
at least one data storing and processing unit performing the analog-to-digital conversion to obtain multiple capacitive sensing values and digitizing the multiple capacitive sensing values to obtain the Boolean true/false results;
at least one human interface electrically connected to the at least one data storing and processing unit to display the capacitive sensing values and the Boolean true/false results.

20. The projected capacitive detecting system as claimed in claim 18, wherein the monitoring module comprises:
at least one data storing and processing unit performing the analog-to-digital conversion to obtain multiple capacitive sensing values and digitizing the multiple capacitive sensing values to obtain the Boolean true/false results;
at least one human interface electrically connected to the at least one data storing and processing unit to display the capacitive sensing values and the Boolean true/false results.
